(19) United States
(12) Patent Application Publication
(10) Pub. No.: US 2004/0088038 A1
(43) Pub. Date: May 6, 2004

(54) Porous metal for drug-loaded stents

(76) Inventors: Houdin Dehnad, El Granada, CA (US); Klaus Kleine, Los Gatos, CA (US); Pamela A. Kramer, San Jose, CA (US); James M. Carlson, Gilroy, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/283,951

(22) Filed: Oct. 30, 2002

(51) Int. Cl. A61F 2/06

(52) U.S. Cl. 623/1.15

(57) ABSTRACT

An intravascular stent for controlled release of therapeutic drugs and for delivery of the therapeutic drugs in localized drug therapy in a blood vessel having a tubular stent member formed of a microcellular porous metal capable of absorbing and releasing therapeutic drugs, wherein a thin layer of a polymeric material is applied to an outer surface of the tubular stent member. A method of making a polymer coated, porous metal stent having sufficient strength and capable of absorbing and releasing therapeutic drugs for the delivery of same in localized drug therapy at an intravascular site is also disclosed herein.

### BACKGROUND OF THE INVENTION

The present invention relates generally to expandable endoprosthesis devices, generally called stents, which are adapted to be implanted into a patient's body lumen, such as a blood vessel, to maintain the patency thereof, and more particularly to polymer coated intravascular stents, formed of a microcellular, porous, metal material, for controlled release and delivery of therapeutic drugs in localized drug therapy in the blood vessel of a patient.

Stents are particularly useful in the treatment and repair of blood vessels after a stenosis has been compressed by percutaneous transluminal coronary angioplasty (PTCA), percutaneous transluminal angioplasty (PTA), or removed by atherectomy or other means, to help improve the results of the procedure and reduce the possibility of restenosis. Stents also can be used to provide primary compression to a stenosis in cases in which no initial PTCA or PTA procedure is performed. While stents are most often used in the procedures mentioned above, they also can be implanted on another body lumen such as the carotid arteries, peripheral vessels, urethra, esophagus and bile duct.

In typical PTCA procedures, a guiding catheter or sheath is percutaneously introduced into the cardiovascular system of a patient through the femoral arteries and advanced through the vasculature until the distal end of the guiding catheter is in the aorta. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to displace the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

In angioplasty procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. Stents are generally cylindrically shaped devices which function to hold open and sometimes expand a segment of a blood vessel or other arterial lumen, such as coronary artery. Stents are usually delivered in a compressed condition to the target location and then are deployed into an expanded condition to support the vessel and help maintain it in an open position. The stent is usually crimped tightly onto a delivery catheter and transported in its delivery diameter through the patient's vasculature. The stent is expandable upon application of a controlled force, often through the inflation of the balloon portion of the delivery catheter, which expands the compressed stent to a larger diameter to be left in place within the artery at the target location. The stent also may be of the self-expanding type formed from, for example, shape memory metals or superelastic nickel-titanum (NiTi) alloys, which will automatically expand from a compressed state when the stent is advanced out of the distal end of the delivery catheter into the body lumen.

The above described, non-surgical interventional procedures, when successful, avoid the necessity for major surgical operations. However, restenosis of blood vessels, such as coronary vessels treated with PTCA or stents (as described above) presents a current clinical challenge. To address this problem, various approaches are being developed to reduce restenosis by locally delivering drugs to the target site of possible restenosis.

Stents are typically formed of a metallic structure to provide the strength required to function as an intravascular device, but have been unable to satisfactorily deliver localized therapeutic pharmacological agents to a blood vessel at the location being treated with the stent. While polymeric materials that can be loaded with and release drugs or other pharmacological treatments can be used for drug delivery, such polymeric materials may not fulfill the structural and mechanical requirements of a stent, especially when the polymeric materials are loaded with a drug, since drug loading of a polymeric material can significantly affect the structural and mechanical properties of the polymeric material. Since it is often useful to provide localized therapeutic pharmacological treatment of a blood vessel at the location being treated with the stent, it would be desirable to provide a porous, metallic stent having sufficient radial strength with the capability of being loaded with therapeutic drugs for the controlled release and delivery of the therapeutic drugs at a specific intravascular site.

Such a metallic component for use in forming intravascular stents capable of carrying and delivering therapeutic drugs can have a microporous structure. Process techniques that have commonly been employed to make metals and polymers microporous include laser drilling of holes in the metal or polymer tubing, and extrusion of the metallic or polymeric material with blowing agents, which may be chemicals or gas, to create cells in the extruded tubing. Laser drilling of such material produces holes in the material, while extrusion with blowing agents commonly results in large non-uniform cells on the order of millimeters in diameter.

Microcellular polymer foams are also known that are characterized by cell sizes in the range of 0.1 to 100 microns, with cell densities in the range of 10⁹ to 10¹⁵ cells per cubic cm. Typically, such microcellular foams exhibit properties comparable or superior to properties of structural foams, and, in some cases to the unfoamed polymer. Suitable microcellular foams are currently preferably produced by exposure of the thermoplastic polymer to supercritical CO₂ fluid under high temperature and pressure to saturate the thermoplastic polymer with the super-critical CO₂ fluid, and then cooling the thermoplastic polymer to foam the amorphous and semi-crystalline thermoplastic polymers. This process is also applicable to fabricate porous, metal foams as used in the present invention. Such suitable microcellular foams can be produced as described in U.S. Pat. No. 4,473,665, which issued Sep. 25, 1984, entitled “Microcellular Closed Cell Foams and Their Method of Manufacture” to Jane E. Martini-Vvedensky et al., commonly owned and assigned to Massachusetts Institute ofTechnology, Cambridge, Mass., the entirety of which is herein incorporated by reference. The foaming process can be carried out on extruded tubing of the proper dimension. The first phase of microcellular foam processing involves dissolving an inert gas, such as nitrogen or CO₂, under pressure into the metal or polymer matrix. The next phase is the rapid creation of microvoids which is initiated by inducing large thermodynamic instability. The thermodynamic instability is induced by quickly decreasing the solubility of the gas in the material by changing the pressure or temperature.

There remains a need for porous metal stents having sufficient radial strength with the capability of being loaded with therapeutic drugs for the controlled release and delivery of the therapeutic drugs at a specific intravascular site. It is desirable that the porous metal stents be formed of a thin wall which will not dramatically increase the delivery profile of the device. Such type of stents should be relatively easy to manufacture as well and should not affect the ability of the stents to be fully deployed within the patient's vasculature. The present invention satisfies these and other needs.

### SUMMARY OF THE INVENTION

The present invention is directed to intraluminal devices and more particularly, porous metal stents for implantation in body vessels which will hold open occluded, weakened or damaged portions of the vessels. The morphology of the microcellular porous metal, including the cell size and porosity of the metal, can be controlled so that the cell sizes can be made very uniform, and can be controlled precisely by changing thermodynamic values like pressure and temperature during formation of the microcellular porous metal. The microcellular porous metal can be formed by a batch process that can be easily controlled and operated, in which extruded tubing can be cut to the desired lengths and then foamed in separate pressure chamber.

The present invention accordingly provides for an intravascular stent for controlled release of therapeutic drugs and for delivery of the therapeutic drugs in localized drug therapy in a blood vessel. In one embodiment, such an intravascular stent is fabricated from a tubular member formed of a microcellular porous metal of a metallic material capable of absorbing and releasing therapeutic drugs, wherein a thin layer of a polymeric material is applied to an outer surface of the tubular member. The microcellular porous metal ranges in thickness from about 0.05 to about 0.5 millimeters. The size of the pores in the metal and the amount of porosity in the metal can be adjusted to accommodate the molecular weight of the particular therapeutic drug. The diameter of the pores or cells of the microcellular porous metal can, for example, be made as small as about a few nanometers to accommodate low molecular weight compounds with molecular weights in the range of 10-1,000 daltons up to large molecular weight compounds with molecular weights in the range of 1,000 to 100,000 daltons, as well as supra molecular structures with molecular weights greater than 100,000 daltons. The size of the pores preferably range from about 0.5 micrometer to about 10 micrometers. The microcellular porous metal is formed from a metallic material such as stainless steel, titanium, tantalum, nickel-titanium, and cobaltchromium. The therapeutic drug carried by the intravascular stent includes antiplatelets, anticoagulants, antifibrins, antithrombins, and antiproliferatives. The polymeric layer applied to the outer surface of the tubular member includes ethyl vinyl alcohol, PBMA, polyurethane, polyethylene, and copolymers and blends thereof, for example, although other similar materials may also be suitable. Examples of supra molecular structures include viral particles used for gene therapy, liposomes, ribozymes, and the like.

In another aspect, the present invention also provides for a method of making an intravascular stent for controlled release of therapeutic drugs and for delivery of the therapeutic drugs in localized drug therapy in a blood vessel. The method entails providing a tubular member formed of a metallic material, treating the tubular member to form microcellular porous metal capable of absorbing and releasing therapeutic drugs in localized drug therapy in a blood vessel, loading the therapeutic drug into the microcellular porous metal, and coating an outer surface of the microcellular porous metal with a polymeric material. The microcellular porous metal can range in thickness from about 0.05 to about 0.5 millimeters, and the diameter of the pores and the amount of porosity in the metal can be adjusted according to the molecular weight of the drug compound. The diameter of the pores or cells of the microcellular porous metal can, for example, be made as small as about a few nanometers to accommodate low molecular weight compounds with molecular weights in the range of 10-1,000 daltons up to large molecular weight compounds with molecular weights in the range of 1,000 to 100,000 daltons, as well as supra molecular structures with molecular weights greater than 100,000 daltons. The size of the pores preferably range from about 0.5 micrometer to about 10 micrometers. The microcellular porous metal is formed from a metallic material selected from the group consisting of stainless steel, titanium, tantalum, nickel-titanium, and cobalt-chromium. The therapeutic drug carried by the intravascular stent is selected from the group consisting of antiplatelets, anticoagulants, antifibrins, antithrombins, and antiproliferatives. The polymeric layer applied to the outer surface of the tubular member includes ethyl vinyl alcohol, PBMA, polyurethane, polyethylene, and copolymers and blends thereof, for example, although other similar materials may also be suitable. Examples of supra molecular structures include viral particles used for gene therapy, liposomes, ribozymes, and the like.

Other features and advantages ofthe present invention will become more apparent from the following detailed description when taken in conjunction with the accompanying exemplary drawings.

### BRIEF DESCRIPTION OF THE DRAWINGS

FIG. 1 is an elevational view, partially in section, of a stent embodying features of the invention which is mounted on a delivery catheter and disposed within a damaged artery.

FIG. 2 is an elevational view, partially in section, similar to that shown in FIG. 1 wherein the stent is expanded within a damaged artery.

FIG. 3A is an elevational view, partially in section, depicting the expanded stent within the artery after withdrawal of the delivery catheter.

FIG. 3B is a plan view of a flattened stent which illustrates the pattern of the stent shown in FIGS. 1-3.

FIG. 4 is an enlarged, transverse cross-sectional view of a porous metal stent similar to that shown in FIG. 3B embodying features of the present invention.

FIG. 5 is an enlarged, cross-sectional view of a portion of the porous metal stent of FIG. 4.

### DETAILED DESCRIPTION OF THE PREFERRED EMBODIMENTS

The present invention is directed to a polymer coated, porous metal stent having sufficient strength and capable of absorbing and releasing therapeutic drugs for the delivery of the same in localized drug therapy at an intravascular site. A method of making an intravascular stent for controlled release and delivery of therapeutic drugs in localized drug therapy in a blood vessel is also disclosed herein.

Turning to the drawings, FIG. 1 depicts a metallic stent 10, incorporating features of the invention, mounted on a catheter assembly 12 which is used to deliver the stent and implant it in a body lumen, such as a coronary artery, carotid artery, peripheral artery, or other vessel or lumen within the body. The stent generally comprises a plurality of radially expandable cylindrical rings 11 disposed generally coaxially and interconnected by undulating links 15 disposed between adjacent cylindrical elements. The catheter assembly includes a catheter shaft 13 which has a proximal end 14 and a distal end 16. The catheter assembly is configured to advance through the patient's vascular system by advancing over a guide wire by any of the well known methods of an over the wire system (not shown) or a well known rapid exchange catheter system, such as the one shown in FIG. 1.

Catheter assembly 12 as depicted in FIG. 1 is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries.

As shown in FIG. 1, a partial cross-section of an artery 24 is shown with a small amount of plaque that has been previously treated by an angioplasty or other repair procedure. Stent 10 of the present invention is used to repair a diseased or damaged arterial wall which may include the plaque 25 as shown in FIG. 1, or a dissection, or a flap which are commonly found in the coronary arteries, carotid arteries, peripheral arteries and other vessels.

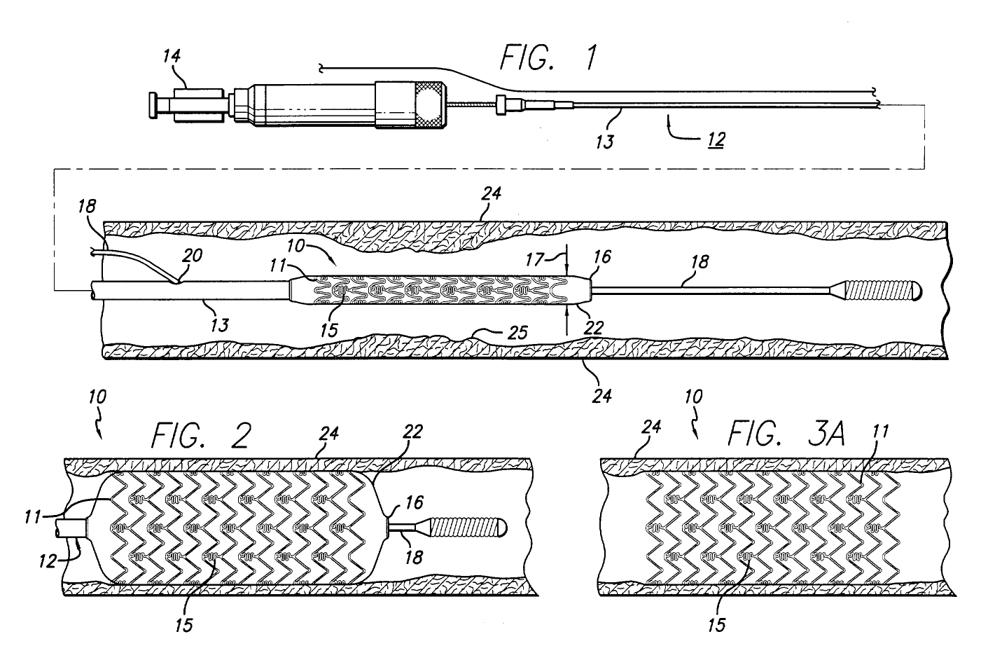

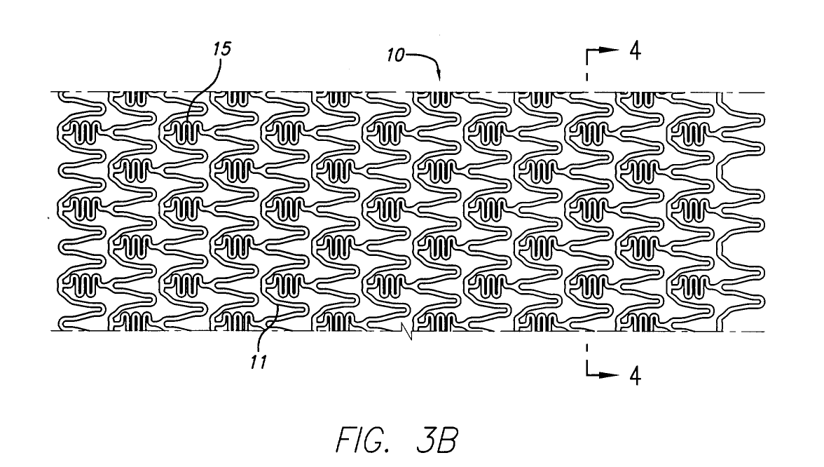

In a typical procedure to implant stent 10, the guide wire 18 is advanced through the patient's vascular system by well known methods so that the distal end of the guide wire is advanced past the plaque or diseased area 25. Prior to implanting the stent, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent is positioned in the target area. The expandable member or balloon 22 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIGS. 2 and 3, the balloon is fully inflated with the stent expanded and pressed against the vessel wall, and in FIG. 3A, the implanted stent remains in the vessel after the balloon has been deflated and the catheter assembly and guide wire have been withdrawn from the patient.

The stent 10 serves to hold open the artery 24 after the catheter is withdrawn, as illustrated by FIG. 3A. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with endothelial cell growth which further minimizes blood flow interference. The undulating portion of the stent provides good tacking characteristics to prevent stent movement within the artery. Furthermore, the closely spaced cylindrical elements at regular intervals provide uniform support for the wall of the artery, and consequently are well adapted to tack up and hold in place small flaps or dissections in the wall of the artery, as illustrated in FIGS. 2 and 3A.

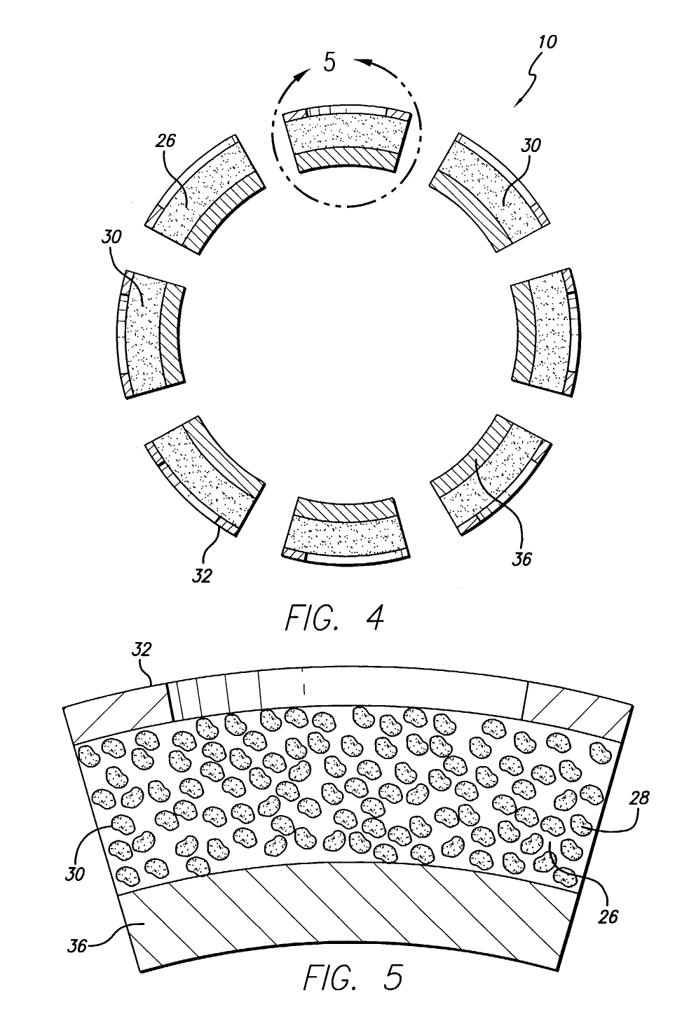

Turning to FIG. 3B, stent 10 is shown in a flattened condition so that the pattern can be clearly viewed, even though the stent is never in this form. The stent is typically formed from a tubular member, however, it can be formed from a flat sheet such as shown in FIG. 4 and rolled into a cylindrical configuration.

The stent patterns shown in FIGS. 1-3 are for illustration purposes only and can vary in size and shape to accommodate different vessels or body lumens. Further, the metallic stent 10 is of a type that can be used in accordance with the present invention.

FIG. 4 illustrates a microcellular porous metal stent 10 formed in accordance with the present invention. In this particular embodiment, the intravascular stent is formed of a metallic tubular member of a microcellular porous metal 26 capable of absorbing and releasing therapeutic drugs 28 for delivery of the drugs in localized drug therapy in a blood vessel. As shown in FIG. 4, a base portion 36 of the tubular stent member is shown as a solid metal. However, it should be appreciated that the entire metallic tubular member can be treated to form microcellular porous metal (not shown). The microcellular porous metal can range in thickness from about 0.05 to about 0.5 millimeters, and the diameter of pores 30 and the amount of porosity in the metal can be adjusted according to the molecular weight of the drug compound. The size of the pores preferably range from about 0.5 micron to about 10 microns. A thin layer of a polymeric material 32 is applied to an outer surface of the tubular member for protection of the therapeutic drug loaded within the microcellular porous metal. The thickness of the polymeric layer ranges from about 0.5 microns to about 20 microns. The thin layer of polymeric material is applied to the outer surface of the tubular member by known methods in the art, such as by coating and dipping.

Microcellular foams are typically characterized by cell sizes or diameters in the range of 0.1 to 100 microns, and cell densities in the range of 10⁹ to 10¹⁵ cells per cubic cm. Typically, microcellular metallic foams exhibit comparable or superior properties to structural polymer foams and the unfoamed polymer. Microcellular foams can be formed based upon the process developed at Massachusetts Institute of Technology and Clarkson University, as outlined in V. Kumar and N. P. Suh, Polym. Eng. Sci., 30, pp.1323-1329 (1990), and C. Wang, K. Cox and G. Campbell, J. Vinyl Additives Tech., 2(2), pp.167-169 (1996). Other various techniques known in the art can be used to fabricate microcellular porous metal. For example, microcellular porous metal can be fabricated by employing the technique of powder technology which involves mixing a select polymer with metal powder and using an injection molding process to shape the tube. Alternatively, an electrolytic process for the deposition of a metal onto a polymer foam precursor by way of electrolytic deposition can be used to fabricate porous metal.

The foaming process can be carried out on metallic preforms such as extruded hypotubing of a desired dimension. The first stage of microcellular foam processing involves dissolving an inert gas, such as nitrogen or CO₂, under pressure into the metallic matrix. The next phase is the rapid creation of microvoids. This is initiated by inducing large thermodynamic instability by quickly decreasing the solubility of the gas in the metal by changing the pressure or temperature.

FIG. 5 illustrates an enlarged view of the microcellular porous metal stent 10 shown in FIG. 4. The diameter of the pores or cells 30 of the microcellular porous metal can, for example, be made as small as about a few nanometers to accommodate low molecular weight compounds with molecular weights in the range of 10-1,000 daltons up to large molecular weight compounds with molecular weights in the range of 1,000 to 100,000 daltons. The metallic material from which the microcellular porous metal can be formed include stainless steel, titanium, tantalum, nickel-titanium, and cobalt-chromium, for example, although other similar materials may also be suitable.

The morphology of the microcellular porous metal, including the cell size and porosity of the metal, can be controlled so that the cell sizes can be made very uniform, and can be controlled precisely by changing thermodynamic variables like pressure and temperature during formation of the microcellular porous metal. The microcellular porous metal can be formed by a batch process that can be easily controlled and operated, in which extruded tubing can be cut to the desired lengths and then foamed in separate pressure chamber.

Examples of therapeutic drugs or pharmacologic compounds that may be loaded into the pores of the microcellular porous metal stent and delivered to the target site in the vasculature include taxol, aspirin, prostaglandins, and the like. Various therapeutic agents such as antithrombogenic or antiproliferative drugs are used to further control local thrombosis. Examples of therapeutic agents or drugs that are suitable for use in accordance with the present invention include sirolimus, everolimus, actinomycin D (ActD), taxol, paclitaxel, or derivatives and analogs thereof. Examples of agents include other antiproliferative substances as well as antineoplastic, antiinflammatory, antiplatelet, anticoagulant, antifibrin, antithrombin, antimitotic, antibiotic, and antioxidant substances. Examples of antineoplastics include taxol (paclitaxel and docetaxel). Further examples of therapeutic drugs or agents include antiplatelets, anticoagulants, antifibrins, antithrombins, and antiproliferatives. Examples of antiplatelets, anticoagulants, antifibrins, and antithrombins include, but are not limited to, sodium heparin, low molecular weight heparin, hirudin, argatroban, forskolin, vapiprost, prostacyclin and prostacyclin analogs, dextran, D-phe-pro-argchloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B(g (an antiplatelet drug from Centocor located in Malvern, Pa). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captopril® (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alphainterferon, genetically engineered epithelial cells, and dexamethasone.

While the foregoing therapeutic agents have been used to prevent or treat restenosis, they are provided by way of example and are not meant to be limiting, since other therapeutic drugs may be developed which are equally applicable for use with the present invention. The treatment of diseases using the above therapeutic agents are known in the art. The calculation of dosages, dosage rates and appropriate duration of treatment are previously known in the art. Furthermore, the therapeutic drugs or agents are loaded at desired concentration levels per methods well known in the art to render the device ready for implantation.

The thin layer of polymer material 32 of the porous, metal stent 10 is selected for its biocompatibility and its permeability to the therapeutic drug 28. The chemical composition of the polymer material and that of the therapeutic drug in combination with the thickness of the polymer material determines the diffusion rate of the therapeutic drug. Further, the release rate of the therapeutic drug can be either controlled by dissolution of the drug and diffusion of the drug through the porous metal or the drug is mixed with a polymer and the release rate of the drug is controlled by the permeability of the drug through the polymer layer.

It should be appreciated that the microcellular, porous metal stent 10 can comprise multiple layers which can be loaded with different therapeutic drugs having varying release rates or a mixture of different therapeutic drugs. A thin layer of polymer material 32 is disposed over the outermost layer of this multiple layer configuration to further control drug elution at the treatment site.

The use of microcellular porous metal for drug loaded stents as in the present invention is advantageous in many respects. With its intricate arrangement of microcellular pores formed throughout the metal stent, the porous metal enables controlled drug loading into the stent and increased storage of the therapeutic drug used for treatment at a particular intravascular site. Additionally, the porous metal provides substantially greater radial strength than drug loaded polymer stents. Other advantages over the prior art include improved protection of the therapeutic drug loaded into the pores of the porous metal stent through use of the thin polymer layer, and ease of manufacturing the porous metal stents by use of metallic materials (i.e., by virtue of the physical properties possessed by metallic materials). The relationship between the material relative density ofthe porous metal and the mechanical properties of the porous metal may be expressed as follows: E_{porous metal}=E_{solid} (P_{porous metal}/ P_{solid})ⁿ where n equals 1.7 . . . . 2, P equals material density, and E equals Young's module. The compressive strength of the porous metal stent has the same relationship.

The present invention also provides a method of making an intravascular stent for controlled release of therapeutic drugs and for delivery of the therapeutic drugs in localized drug therapy in a blood vessel. The method consists of providing a tubular member formed of a metallic material. The tubular member is treated to form porous metal 26 capable of absorbing and releasing therapeutic drugs 28 in localized drug therapy in a patient's blood vessel (FIGS. 4 and 5). Treatment of the tubular member to form the microcellular porous metal can be formed pursuant to one of the earlier mentioned techniques known in the art. Following the formation of the microcellular porous metal, the porous metal is preferably laser cut into a desired shape to form the tubular stent member 10. Once the stent is electropolished, the therapeutic drug is ready to be loaded into the microcellular porous metal. A thin coating of polymeric material 32 is used to coat an outer surface ofthe microcellular porous metal stent.

In use, the stent is deployed using conventional techniques. Once in position, the therapeutic drug gradually diffuses into adjacent tissue at a rate dictated by the parameters associated with the polymer coat layer. The total dosage that is delivered is of course limited by the total amount of the therapeutic drug that had been loaded within the porous metal stent. The therapeutic drug is selected to treat the deployment site and/or locations downstream thereof. For example, deployment in the carotid artery will serve to deliver such therapeutic drug to the brain.

The aforedescribed illustrative stent 10 of the present invention and similar stent structures can be made in many ways. Following treatment of the tubular member to form the microcellular porous metal stent in accordance with one of the earlier mentioned techniques known in the art, one method of making the stent rings 11 is to cut a thin-walled tubular member, such as stainless steel tubing to remove portions of the tubing in the desired pattern for the stent, leaving relatively untouched the portions of the metallic tubing which are to form the rings. In accordance with the invention, it is preferred to cut the tubing in the desired pattern using a machinecontrolled laser which process is well known in the art.

After laser cutting, the stent rings are preferably electrochemically polished in an acidic aqueous solution such as a solution of ELECTRO-GLO #300, sold by the ELECTRO-GLO Co., Inc. in Chicago, Ill., which is a mixture of sulfuric acid, carboxylic acids, phosphates, corrosion inhibitors and a biodegradable surface active agent. The bath temperature is maintained at about 110-135° F. and the current density is about 0.4 to about 1.5 amps per square inch. Cathode to anode area should be at least about two to one.

The foregoing laser cutting process to form the cylindrical rings 11 can be used with metals other than stainless steel including cobalt-chromium, titanium, tantalum, nickel-titanium, and other biocompatible metals suitable for use in humans, and typically used for intravascular stents. Further, while the formation of the cylindrical rings is described in detail, other processes of forming the rings are possible and are known in the art, such as by using chemical etching, electronic discharge machining, stamping, and other processes.

While the invention has been described in connection with certain disclosed embodiments, it is not intended to limit the scope of the invention to the particular forms set forth, but, on the contrary it is intended to cover all such alternatives, modifications, and equivalents as may be included in the spirit and scope of the invention as defined by the appended claims.

What is claimed:

1. An intravascular stent for controlled release of therapeutic drugs and for delivery of the therapeutic drugs in localized drug therapy in a blood vessel, comprising:
a tubular member formed of a microcellular, porous metal material capable of absorbing and releasing therapeutic drugs, wherein a thin layer of a polymeric material is applied to an outer surface of the tubular member.

2. The intravascular stent of claim 1, wherein the microcellular, porous metal has a thickness in the range of from about 0.05 up to about 0.5 millimeters.

3. The intravascular stent of claim 1, wherein the size of the pores in the microcellular metal and the amount of porosity in the microcellular metal is adjusted to accommodate the molecular weight of the therapeutic drug.

4. The intravascular stent of claim 1, wherein the pores of the microcellular metal have a size in the range of from about 0.5 micron up to about 10 microns.

5. The intravascular stent of claim 1, wherein the diameter of the pores of the microcellular metal is formed to accommodate a compound having a molecular weight in the range of from about 10 daltons up to about 1,000,000 daltons.

6. The intravascular stent of claim 1, wherein the microcellular, porous metal is formed from a material selected from the group consisting of stainless steel, titanium, tantalum, nickel-titanium, cobalt-chromium, and alloys thereof.

7. The intravascular stent of claim 1, wherein the therapeutic drug is selected from the group consisting of antiplatelets, anticoagulants, antifibrins, antithrombins, and antiproliferatives.

8. The intravascular stent of claim 1, wherein the polymeric layer includes ethyl vinyl alcohol, PBMA, polyurethane, polyethylene, and copolymers and blends thereof.

9. The intravascular stent of claim 1, wherein the microcellular, porous metal comprises multiple layers having therapeutic drug loaded therein.

10. An intravascular stent for controlled release of therapeutic drugs and for delivery of the therapeutic drugs in localized drug therapy in a blood vessel, comprising:
a tubular member formed of a microcellular, porous metal foam capable of absorbing and releasing therapeutic drugs, wherein a thin layer of a polymeric material is applied to an outer surface of the tubular member.

11. A method of making an intravascular stent for controlled release of therapeutic drugs and for delivery of the therapeutic drugs in localized drug therapy in a blood vessel, comprising:
providing a tubular member formed of a metallic material;
treating the tubular member to form microcellular, porous metal capable of absorbing and releasing therapeutic drugs in localized drug therapy in a blood vessel;
laser-cutting the microcellular, porous metal into a desired pattern to form the stent;
electropolishing the microcellular, porous metal stent;
loading the therapeutic drug into the microcellular, porous metal stent; and
coating an outer surface of the microcellular, porous metal stent with a polymeric material.

12. The method of claim 11, further comprising adjusting the size of the pores in the microcellular metal and the amount of porosity in the microcellular metal to accommodate the molecular weight of the therapeutic drugs.

13. The method of claim 11, wherein the pores of the microcellular metal have a size in the range of from about 0.5 micron up to about 10 microns.

14. The method of claim 11, wherein the microcellular, porous metal has a thickness in the range of from about 0.05 up to about 0.5 millimeters.

15. The method of claim 11, wherein the diameter of the pores of the microcellular metal is formed to accommodate a compound having a molecular weight in the range of from about 10 daltons up to about 1,000,000 daltons.

16. The method of claim 11, wherein the microcellular, porous metal is formed from a material selected from the group consisting of stainless steel, titanium, tantalum, nickel-titanium, cobalt-chromium, and alloys thereof.

17. The method of claim 11, wherein the therapeutic drug is selected from the group consisting of antiplatelets, anticoagulants, antifibrins, antithrombins, and antiproliferatives.

18. The method of claim 11, wherein the coating of the outer surface of the microcellular, porous metal includes ethyl vinyl alcohol, PBMA, polyurethane, polyethylene, and copolymers and blends thereof.

19. The method of claim 11, wherein the microcellular, porous metal is formed by use of powder technology.

20. The method of claim 11, wherein the microcellular, porous metal is formed by foaming the tubular member.
